(19) United States
(12) Patent Application Publication
(10) Pub. No.: US 2015/0099307 A1
(43) Pub. Date: Apr. 9, 2015

(54) GLUCOSE TEST STRIP AUTHENTICATION USING INK

(71) Applicant: Texas Instruments Incorporaated, Dallas, TX (US)

(72) Inventor: EDUARDO BARTOLOME, DALLAS, TX (US)

(21) Appl. No.: 14/478,781

(22) Filed: Sep. 5, 2014

Related U.S. Application Data
(60) Provisional application No. 61/887,483, filed on Oct. 7, 2013.

(51) Int. Cl. G01N 33/66

(52) U.S. Cl. CPC G01N 33/66
USPC 436/95; 422/82.05

(57) ABSTRACT

A method of authenticating includes providing a glucose test strip having a top surface with a reagent thereon that has a structure which chemically reacts with glucose and an anti-counterfeiting identification feature (identification feature) including at least one ink. The glucose test strip is inserted into a glucose meter. The glucose meter includes a light source positioned to shine light on the ink after the inserting, a photodetector positioned to detect a reflected or transmitted signal after interaction with the ink, and stored information that identifies the identification feature. A processor implementing an algorithm automatically analyzes the reflected or transmitted signal by reference to the stored information to determine whether the glucose test strip is authentic.

### CROSS REFERENCE TO RELATED APPLICATIONS

This application claims the benefit of Provisional Application Ser. No. 61/887,483 entitled “BLOOD GLUCOSE METER STRIP AUTHENTICATION METHOD THROUGH INK”, filed Oct. 7, 2013, which is herein incorporated by reference in its entirety.

### FIELD

Disclosed embodiments relate to authentication of glucose test strips.

### BACKGROUND

Individuals suffering from diabetes typically monitor their blood glucose concentrations several times per day, generally at home using disposable test strips having a reagent disposed thereon that chemically reacts with a biological fluid such as blood. Such test strips work in conjunction with a blood glucose meter. Blood glucose meters are generally small, portable, and easy to use.

Conventional blood glucose meters work with electrochemical test strips which have electrodes where a precise bias voltage is applied using a digital-to-analog converter (DAC), and a current proportional to the glucose in the blood is measured as a result of the electrochemical reaction of the glucose with the reagent on the test strip. The test strip is mated to a glucose test meter such that the test meter measures the reaction between the blood analyte and the reagent to determine the glucose concentration of the analyte. For electrochemically-based test strips, the electrical signal is transferred to the meter through a pair of electrical contact pads on the test strips which contacts within the meter strip port connector. There can be one or more channels, and the current signal measured is generally converted to a voltage by a transimpedance amplifier (TIA) for measurement with an analog-to-digital converter (ADC).

Some blood glucose meters instead work with optical-reflectometry test strips which use color to determine the glucose concentration in the blood. Typically, a calibrated current passes through two light-emitting diodes (LEDs) or lasers that alternately flash onto the colored test strip. A photodiode senses the reflected light intensity, which is dependent on the color of the test strip, which, in turn, is dependent on the amount of glucose in the blood. The photodiode current is usually converted to a voltage by a TIA for measurement with an ADC.

One problem for blood glucose test strips is counterfeiting. Counterfeit test strips can provide incorrect blood glucose values, either too high or too low, which can result in a patient taking either too much or too little insulin. Counterfeit test strips can thus lead to serious injury or death. Some glucose test strips vendors have tried using different approaches on or in the strip including Electrically Erasable Programmable Read-Only Memory (EEPROM) and Radio-Frequency Identification (RFID), which are generally too expensive to be widely applied because the test strips need to be low cost since they are disposable.

### SUMMARY

This Summary is provided to introduce a brief selection of disclosed concepts in a simplified form that are further described below in the Detailed Description including the drawings provided. This Summary is not intended to limit the claimed subject matter's scope.

Disclosed embodiments describe low-cost methods to authenticate blood glucose meter (BGM) test strips. One disclosed embodiment is a method of authenticating which includes providing a glucose test strip having a top surface with a reagent thereon (or therein) that has a structure which chemically reacts with glucose and an anti-counterfeiting identification feature (identification feature) comprising at least one ink. The glucose test strip is inserted into a glucose meter. The glucose meter includes a light source positioned to shine light on the ink after the inserting, a photodetector is positioned to detect a reflected or a transmitted signal after interaction with the ink, and stored information that identifies the identification feature. A processor implementing an algorithm automatically analyzes the reflected or transmitted signal by reference to the stored information to determine whether the glucose test strip is authentic.

### BRIEF DESCRIPTION OF THE DRAWINGS

Reference will now be made to the accompanying drawings, which are not necessarily drawn to scale, wherein:

FIG. 1A is a depiction of an example glucose test strip having a top surface with a reagent thereon that has a structure which chemically reacts with glucose and an identification feature comprising at least one ink on an opposite side of the test strip alongside an example optical transmission or reflection-based BGM, according to an example embodiment.

FIG. 1B is a depiction of an example glucose test strip having a top surface with a reagent thereon that has a structure which chemically reacts with glucose connected to leads and an identification feature comprising at least one ink on an opposite side of the test strip alongside an example electrochemical BGM, according to an example embodiment.

FIG. 2 is a side cross sectional view of an example blood glucose measurement system having strip authentication including a glucose test strip having a top surface with a reagent thereon that has a structure which chemically reacts with glucose and an identification feature comprising at least one ink on an opposite side of the test strip inserted inside an example optical transmission-based BGM, according to an example embodiment.

FIG. 3 is a side cross sectional view of an example blood glucose measurement system having strip authentication including a glucose test strip having a top surface with a reagent thereon that has a structure which chemically reacts with glucose and an identification feature comprising at least one ink on an opposite side of the test strip inserted inside an example optical reflection-based BGM, according to an example embodiment.

### DETAILED DESCRIPTION

Example embodiments are described with reference to the drawings, wherein like reference numerals are used to designate similar or equivalent elements. Illustrated ordering of acts or events should not be considered as limiting, as some acts or events may occur in different order and/or concurrently with other acts or events. Furthermore, some illustrated acts or events may not be required to implement a methodology in accordance with this disclosure.

In one disclosed embodiment, a glucose test strip is provided, where the ink on the glucose test strip is a fluorescent or phosphorescent ink that has at least one distinguishing identification feature that comprises a characteristic wavelength shift, or an amplitude of a reflected or transmitted signal as a function of time. The ink can be printed to be configured in a characteristic pattern. For example, an example characteristic pattern comprises a bar code pattern. The ink coating on the test strip is generally only on the tip of the test strip, and the test strip is read it once it gets inserted in the BGM, typically before the sample (e.g., blood) is drawn by the patient.

The ink properties being known including its distinguishing identification characteristic(s) and stored in a memory of a BGM allows distinguishing a counterfeit test strip from an authentic test strip. An “ink” as used herein is based on its conventional definition being a liquid or paste that contains pigments or dyes used to color a surface to produce an image, text, or design. The ink can be a complex medium, comprising solvents, pigments, dyes, resins, lubricants, solubilizers, surfactants, particulate matter, fluorescers, as well as other materials. The components of inks generally serve many purposes including the ink's carrier, colorants, and other additives affect the flow and thickness of the ink and its appearance when dry. One vendor that supplies inks that may be used for disclosed embodiments is Authentix, Inc.

Disclosed embodiments include methods of authenticating glucose test strips. A glucose test strip is provided having a top surface with a reagent thereon or therein that has a structure which chemically reacts with glucose and an identification feature comprising at least one ink. The glucose test strip is inserted into a glucose meter. The glucose meter includes a light source positioned to shine light on the ink after the test strip is inserted, a photodetector positioned to detect a reflected or transmitted signal after interaction with the ink, and stored information that identifies the identification feature. The reflected or transmitted signal is automatically analyzed using a processor implementing an algorithm by reference to the stored information to determine whether the glucose test strip is authentic (i.e., not counterfeit). The glucose meter can be programmed to not render any glucose measurements unless the glucose test strip is determined to be authentic.

The distinguishing identification characteristic can comprise a photoemissions characteristic of the ink, such as a characteristic wavelength shift (relative to a wavelength of the incident light), or an amplitude of a reflected or transmitted signal as a function of time. As noted above, the ink can be a fluorescent or phosphorescent ink.

The BGM can be an electrochemical glucose meter including at least a pair of electrical contact pads for contacting leads on the glucose test strip. In another embodiment, the BGM is an optical-based glucose meter, including a light source such as a light-emitting diode (LED) or a laser, and a photodetector, being either reflectometry-based or a transmission-based BGM. The same light source and photodetector used for authentication can be used for the glucose measurement as well as some other electronics.

Regarding a typical user use sequence, a patient inserts the glucose test strip in the BGM, where the BGM may perform some calibration of the empty strip that generally happens transparent to the patient. The patient then pricks his or her finger (e.g., with a lancet), and finally approaches the test strip to the flowing blood from the finger to collect a sample and measure it. The patient inserts the strip in the BGM, where the same side of the test strip can have an ink identification feature thereon. Inside the BGM, there is a light source and a photodetector, such as a photodiode/LED pair. The light source (e.g., LED) illuminates the sample and the photodetector measures the fluorescence of phosphorous response, such as with a given wavelength and time pattern. If the test strip is a counterfeit, the BGM can display an error on its display before the patient pricks his or her finger.

Some end applications may need coding in the test strip to represent a large number, such as to represent a calibration code, serial number or a manufacturing date. Although the properties of the ink may allow for some variations, such variations may not be able to represent that number, or the implementation may become complex. Therefore, in one embodiment an ink pattern can be created having a variation along the insertion direction of the test strip, such as a bar code. The pattern can be read in portions by the individual light source/photodetector as the test strip is inserted into the BGM.

In order to conserve power, the light source/photodetector can be off most of the time, and then powered up when the test strip is inserted. A conventional approach is to add a switch, either an off-the shelf switch that is closed when the test strip tip enters the BGM, or by having an electrical conductor on the test strip that shorts two terminals at the orifice of the BGM, such as on the board in one embodiment, effectively closing the switch. In either case, the switch can trigger an interrupt through the processor's general-purpose input/output (GPIO) pin, which in turn, can turn on the light source/photodetector just in time to read the pattern as it slides in front of them.

Ink-based identification features can be formed using any suitable ink-dispensing technique including, for example, inkjet printing, thermal transfer, syringe coating, slot coating, graviere coating, flexographic coating or screen printing techniques. A typical, but non-limiting, thickness for the identification feature(s) is in the range of 1 micron to 10 microns.

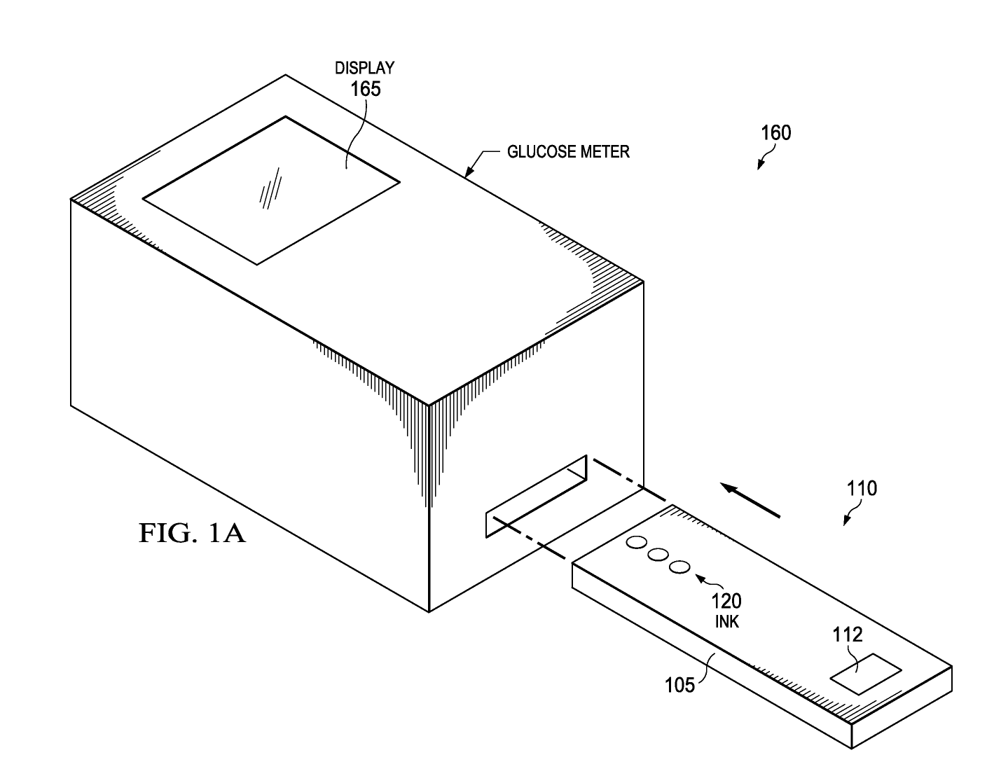

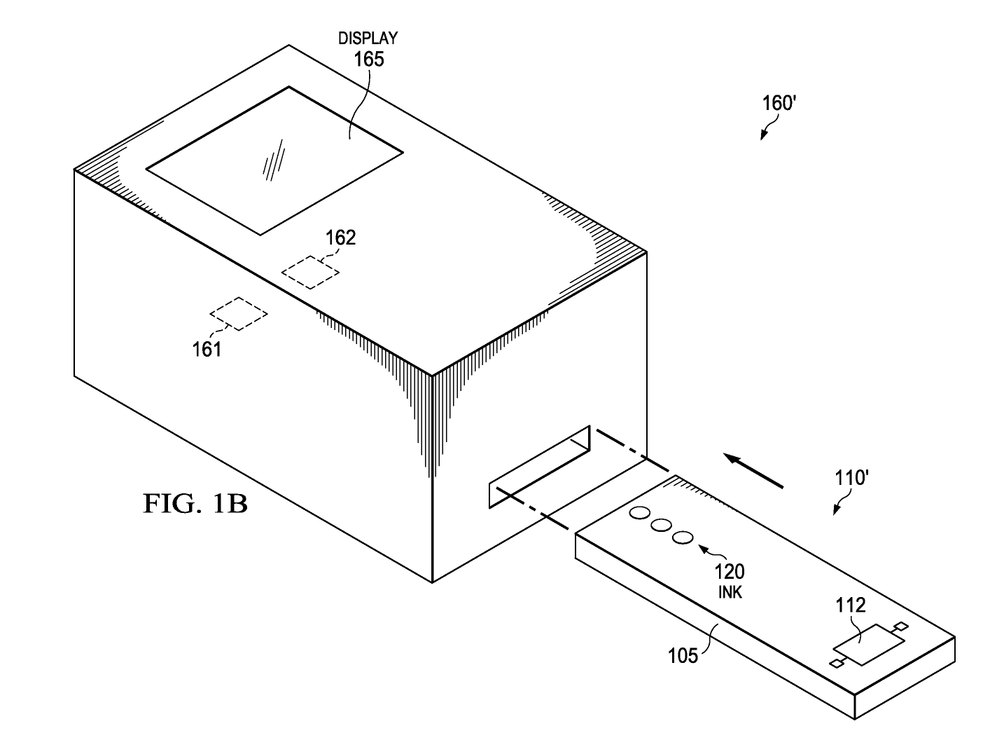

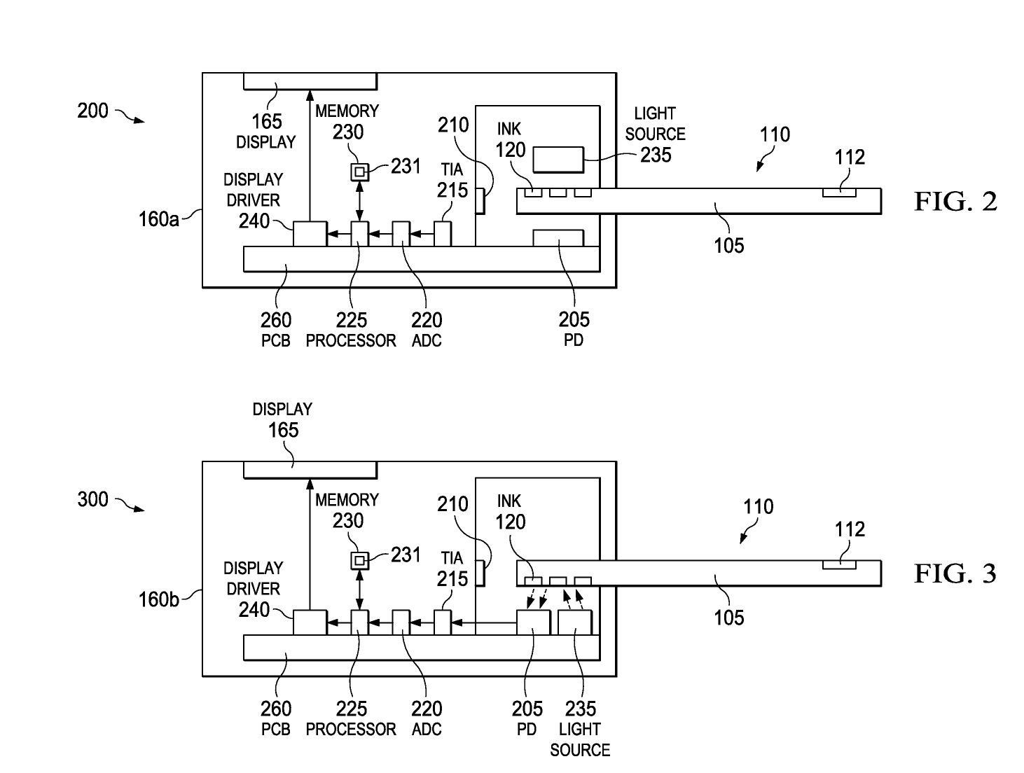

FIG. 1A is a depiction of an example glucose test strip 110 comprising a substrate 105 having a top surface of the substrate with a reagent 112 thereon or therein (such as within a well on the top surface) that has a structure which chemically reacts with glucose and an identification feature comprising at least one ink 120 on an opposite side of the test strip 110 alongside an example optical transmission or reflection-based BGM 160, according to an example embodiment. The BGM 160 shown as a “glucose meter” includes a display 165, and includes an opening to allow insertion of the test strip 110. FIGS. 2 and 3 described below show example optical portions (optical transmission-based and reflection-based, respectively) and electronics portions for performing strip authentication. The optical portion (light source and photodetector) and the electronics portion (transimpedance amplifier (TIA), analog-to-digital converter (ADC) and processor) of the BGM can be used for both performing strip authentication and for the optical glucose measurement.

FIG. 1B is a depiction of an example glucose test strip 110′ having a top surface with a reagent thereon that has a structure which chemically reacts with glucose connected to leads and an identification feature comprising at least one ink 120 on an opposite side of the test strip alongside an example electrochemical BGM 160′, according to an example embodiment. The electrochemical BGM 160′ includes at least a pair of electrical contact pads 161 and 162 for contacting leads on the glucose test strip. The glucose test strip 110′ may have many more than the pair of contacts shown. Some of the electronics portion (TIA, ADC and processor) of the BGM 160′ can be used for both performing strip authentication and for the electrochemical glucose measurement.

FIG. 2 is a side cross sectional view of an example blood glucose measurement system 200 having strip authentication including a glucose test strip 110 having a top surface with a reagent thereon that has a structure which chemically reacts with glucose and an identification feature comprising at least one ink 120 on an opposite side of the test strip inserted inside an example optical transmission-based BGM 160a, according to an example embodiment. In this embodiment the substrate generally comprises an optically transmissive material in a wavelength range from 200 nm to 2 μm to enable light transmission through the substrate to reach the photodetector 205 with less than 50% attenuation. A test strip stopper 210 is shown that controls the fully inserted position of the glucose test strip 110.

BGM 160a includes a printed circuit board (PCB) 260 that is shown having a photodetector (PD) 205 coupled to a TIA 215 coupled to an ADC 220. PD 205 can comprise photodiodes or a charge-coupled device (CCD). In this embodiment the substrate for the glucose test strip 110 is optically transparent for the wavelength provided by the light source 235. As described above, the optical portion (light source 235 and photodetector 205) and the electronics portion (TIA 215, ADC 220 and processor 225) of the BGM 160a can be used for both performing strip authentication and for the optical glucose measurement.

An output of the ADC 220 is coupled to an input of a processor 225 that has an associated memory 230 that stores stored information that identifies the identification feature and a disclosed algorithm shown as 231 that is configured when implemented by the processor 225 to automatically analyzing the transmitted signal from light source 235 by reference after interacting with the ink 120 to the stored information to determine whether the glucose test strip 110 is authentic. The light source 235 can comprise a LED, or a laser (e.g., a vertical-cavity surface-emitting laser (VCSEL)). The output of the processor 225 is coupled to an input of a display driver 240 which is coupled to drive the display 165, such as to indicate whether the glucose test strip 110 is authentic.

FIG. 3 is a side cross sectional view of an example blood glucose measurement system 300 having strip authentication including a glucose test strip 110 having a top surface with a reagent thereon that has a structure which chemically reacts with glucose and an identification feature comprising at least one ink 120 on an opposite side of the test strip inserted inside an example optical reflection-based BGM 160b, according to an example embodiment. In this embodiment both the light source 235 and the PD 205 are provided on the PCB 260. As with other disclosed BGM's, the optical portion (light source 235 and photodetector 205) and the electronics portion (TIA 215, ADC 220 and processor 225) of BGM 160b can be used for both performing strip authentication and for the optical glucose measurement.

Those skilled in the art to which this disclosure relates will appreciate that many other embodiments and variations of embodiments are possible within the scope of the claimed invention, and further additions, deletions, substitutions and modifications may be made to the described embodiments without departing from the scope of this disclosure. For example, disclosed embodiments may also be used to authenticate test strips for testing levels of materials other than glucose, such as cholesterol, pathogenic bacteria or generally any other strip based assay.

1. A method of authenticating, comprising:
providing a glucose test strip having a top surface with a reagent thereon that has a structure which chemically reacts with glucose and an anti-counterfeiting identification feature (identification feature) comprising at least one ink;
inserting said glucose test strip into a glucose meter,
wherein said glucose meter includes a light source positioned to shine light on said ink after said inserting, a photodetector positioned to detect a reflected or transmitted signal after interaction with said ink, and stored information that identifies said anti-counterfeiting identification feature, and
using a processor implementing an algorithm, automatically analyzing said reflected or said transmitted signal by reference to said stored information to determine whether said glucose test strip is authentic.

2. The method of claim 1, wherein said glucose meter is programmed to not render any glucose measurements unless said glucose test strip is determined to be authentic.

3. The method of claim 1, wherein said identification feature comprises photoemission characteristics of said ink.

4. The method of claim 1, wherein said ink is a fluorescent or phosphorescent ink, and said identification feature comprises a characteristic wavelength shift.

5. The method of claim 1, wherein said identification feature comprises an amplitude of said reflected or said transmitted signal as a function of time.

6. The method of claim 1, wherein said glucose meter is an electrochemical glucose meter including at least a pair of electrical contact pads for contacting leads on said glucose test strip.

7. The method of claim 1, wherein said glucose meter is an optical-reflectometry glucose meter or an optical-transmission-based glucose meter, further comprising using said light source and said photodetector for glucose measurements involving said reagent.

8. The method of claim 1, wherein said ink is configured in a characteristic pattern.

9. The method of claim 8, wherein said characteristic pattern comprises a bar code pattern.

10. A glucose test strip, comprising:
a substrate having a top surface;
a reagent which has a structure that chemically reacts with glucose, and
at least one anti-counterfeiting identification feature (identification feature) comprising at least one ink on said top surface.

11. The glucose test strip of claim 10, wherein said ink is a fluorescent or phosphorescent ink, and said identification feature comprises a characteristic wavelength shift.

12. The glucose test strip of claim 10, wherein said reagent and said ink are on opposite sides of said glucose test strip.

13. The glucose test strip of claim 10, wherein said ink is configured in a bar code pattern.

14. The glucose test strip of claim 10, wherein said substrate comprises an optically transmissive material in a wavelength range from 200 nm to 2 μm.

15. A blood glucose measurement system having strip authentication, comprising:
a glucose test strip having a top surface with a reagent thereon that has a structure which chemically reacts with glucose and an anti-counterfeiting identification feature (identification feature) comprising at least one ink configured for inserting into a glucose meter,
wherein said glucose meter includes a light source positioned to shine light on said ink after said inserting, a photodetector positioned to detect a reflected or transmitted signal after interaction with said ink, and stored information that identifies said identification feature, and
a processor implementing an algorithm for automatically analyzing said reflected or said transmitted signal by reference to said stored information to determine whether said glucose test strip is authentic.

16. The blood glucose measurement system of claim 15, wherein said glucose meter is programmed to not render any glucose measurements unless said glucose test strip is determined to be authentic.

17. The blood glucose measurement system of claim 15, wherein said ink is a fluorescent or phosphorescent ink, and said identification feature comprises a characteristic wavelength shift.

18. The blood glucose measurement system of claim 15, wherein said glucose meter is an electrochemical glucose meter including at least a pair of electrical contact pads for contacting leads on said glucose test strip.

19. The blood glucose measurement system of claim 15, wherein said glucose meter is an optical-reflectometry glucose meter or an optical-transmission-based glucose meter that uses said light source and said photodetector for shining said light on said ink and for glucose measurements involving said reagent.

20. The blood glucose measurement system of claim 15, wherein said light source and said photodetector are further configured for glucose measurements involving said reagent.
